(19) United States
(12) Patent Application Publication
(10) Pub. No.: US 2010/0159501 A1
(43) Pub. Date: Jun. 24, 2010

(54) METHOD FOR QUANTIFICATION OF CELLULAR SPHINGOLIPIDS

(75) Inventors: Mark R. Emmett, Monticello, FL (US); Huan He, Tallahassee, FL (US); Alan G. Marshall, Tallahassee, FL (US)

Correspondence Address: SUTHERLAND ASBILL & BRENNAN LLP, 999 PEACHTREE STREET, N.E., ATLANTA, GA, 30309, US

(73) Assignee: FLORIDA STATE UNIVERSITY RESEARCH FOUNDATION, Tallahassee, FL (US)

(21) Appl. No.: 12/646,352

(22) Filed: Dec. 23, 2009

Related U.S. Application Data
(60) Provisional application No. 61/140,674, filed on Dec. 24, 2008.

(51) Int. Cl. C12Q 1/02; C07C 229/22; C07C 229/08

(52) U.S. Cl. 435/29; 562/567; 562/553

(57) ABSTRACT

A method is provided for quantifying endogenous sphingolipids in a biological system. The method includes preparing one or more isotope labeled amino acids; introducing the isotope labeled amino acids into a biological system; extracting and separating a sphingolipid-containing fraction from the biological system; and quantifying the amount of endogenous sphingolipids in the biological system. The isotope-labeled amino acid may include a non-essential amino acid, and the method may further include adding an amino acid synthesis inhibitor into the biological system. Systems and kits for quantifying endogenous sphingolipids also are disclosed.

### CROSS-REFERENCE TO RELATED APPLICATIONS

The present application claims benefit to U.S. Provisional Application No. 61/140,674, filed Dec. 24, 2008. This application is incorporated herein by reference.

### FEDERALLY SPONSORED RESEARCH

This work was supported by the National Science Foundation Grant No. DMR-0654118. The government may have certain rights in the invention.

### BACKGROUND

This invention relates to the relative quantification of cellular sphingolipids. Relative quantification of cellular sphingolipids during various disease states and treatment may provide insight into the underlying biological process and thus aid in discovery of new biomarkers and therapeutic targets for treating specific disease states.

Sphingolipids share a common structural motif of a sphingoid backbone. Most sphingolipids consist of a polar head group attached to a non-polar ceramide tail. These compounds are the major components of the cell membrane (known as the lipid bilayer), and are positioned in the membrane with the head groups pointing either outward toward the aqueous extracellular environment (extracellular) or inward toward the aqueous cytoplasmic environment (intracellular). These compounds can vary greatly in the composition of their polar head groups and the composition of their non-polar ceramide tails.

Sphingolipids play important roles in cell-cell recognition, control of cell division, differentiation, and adhesion. Several sphingolipids have been identified as biomarkers in a variety of diseases, including cancer, diabetes, multiple sclerosis, Tay Sachs disease and others. For example, long chain sulfatides (sulfate-containing sphingolipids) are a major part of the myelin sheath and can be used as a biomarker for monitoring the progression of multiple sclerosis. The cellular sphingolipid profile changes during the cell cycle, during apoptosis (programmed cell death) and in different disease states.

Glioblastoma multiforme (GM) brain tumors are among the most devastating of all primary brain tumors. GM are nearly uniformly fatal, with median survival between 9 and 12 months from initial diagnosis (CBTRUS, 2002-2003). Their highly invasive behavior makes complete surgical removal virtually unachievable. The cornerstone of treatment for GM still includes surgery, radiation therapy (RT), and chemotherapy, but these interventions are still largely ineffective, and the majority of patients experience progression or recurrence (ABTA, 2004). The severity of these tumors necessitates a better understanding of the disease on a molecular level. Previously, it was reported that reduced levels of a sugar-binding protein, galectin-1 (gal1), were correlated with decreased invasiveness and greater tendency toward apoptosis (tumor cell death) (Puchades, et al., J. Proteome Res. 2007, 6, 869-875). Gal1 was identified as being a potential therapeutic target for the treatment of GM. Gal1 specifically binds the sugar, beta-galactose, when it is the terminal sugar on both proteins and on lipids.

Although methods exist for work at the protein level, there remains a need to develop a method to work on the lipid level. Accordingly, the applicants previously developed an assay (the “Lipid Method”) to isolate, detect, and identify polar lipids from GM samples under various stages of treatment to gain a better understanding of the glioblastoma system at the molecular level. (He, et al. Analytical Chemistry, 2007, 79: 8423-8430). These methods enabled the analysis and identification of ˜1,000 lipids that varied by polar head group and non-polar tails (length, degree of saturation [double bonds] and modification [esp. hydroxylation]). While others focused on either the polar head group or small subsets of lipid families (e.g. phospholipids), the Lipid Method provided for the ability to monitor changes in hundreds of polar lipids in these brain tumor cell lines under a variety of treatment regimes.

Although the Lipid Method provides a qualitative analysis by identifying the type of sphingolipid present in a sample, it is only semi-quantitative. Many factors keep the current method from being quantitative, including, but not limited to chemical complexity of the sample, which results in masking of some components by others; and low abundance of signal, which results in incomplete statistical sampling. Methods for true quantitation of the sphingolipids would aid in understanding the molecular changes in cancer cells.

Current methods for quantification of polar lipids have used a series of externally administered internal standards of various molecular weights or chemical modification of the sphingolipids that are already present. Although these methods improve quantitation, they are merely semi-quantitative. The addition of external standards does not take into account the biological modifications that occur to the ceramide tail (hydroxylation, degree of saturation, etc.) and the available external standards are limited. Chemical modifications tend not to go to 100% completion and can vary due to sample composition and thus are not truly quantitative. Thus, there exists a need for a method for the true, or at least improved, quantitation of biological sphingolipids.

### SUMMARY

A method of quantifying endogenous sphingolipids in a biological system is provided comprising: preparing one or more isotope labeled amino acids; introducing the isotope labeled amino acids into a biological system; extracting and separating a sphingolipid-containing fraction from the biological system; and quantifying the amount of endogenous sphingolipids in the biological system.

Also provided are kits and systems for the quantification of cellular sphingolipids by metabolic labeling. In one embodiment, a kit is provided for the quantification of cellular sphinglipids by metabolic labeling comprising one or more isotope-labeled amino acids.

### BRIEF DESCRIPTION OF THE DRAWINGS

FIG. 1 is a schematic illustration of a metabolic pathway for a sphingolipid.

FIG. 2 is a schematic illustration of a method for quantitation of sphingolipids.

FIGS. 3A and 3B are mass spectra of GM2α and GD2, respectively, obtained according to one embodiment.

### DETAILED DESCRIPTION

A method has been developed for the quantification of sphingolipids by metabolic labeling. The method provides improved analytical means for understanding important biological processes (e.g., the diagnosis and treatment of diseases). Generally described, the method comprises preparing one or more isotope-labeled amino acids, introducing the one or more isotope-labeled amino acids into a biological system, extracting a sphingolipid-containing fraction from the biological system, and analyzing the sphingolipid-containing fraction.

A “sphingolipid,” as used herein, is any of a group of lipids that yields sphingosine or its derivatives upon hydrolysis. Non-limiting examples of sphingolipids include sphingomyelins and glycosphingolipids such as cerbrosides, gangliosides, and sulfatides.

In one embodiment, the method comprises preparing isotope-labeled amino acids for introduction into a biological system. The isotope-labeled amino acid may comprise an essential amino acid or a non-essential amino acid. An “essential amino acid,” as used herein, is an amino acid that an organism is incapable of independently synthesizing and which is required for protein synthesis. Non-limiting examples of essential amino acids include arginine, histidine, methionine, threonine, valine, isoleucine, phenylalanine, tryptophan, leucine, and lysine. A “non-essential amino acid,” as used herein, is an amino acid that an organism is capable of synthesizing independently. Non-limiting examples of non-essential amino acids include serine, glycine, alanine, asparagine, aspartate, cysteine, glutamate, glutamine, proline, and tyrosine.

Methods for preparing isotope-labeled amino acids are known in the art. The isotope labeling of the amino acid can be done in such a way that the chemical behavior of the amino acid is not modified, thereby permitting tracking of the path of the amino acid through the sphingolipid biosynthetic pathway in the biological system. For example, an amino acid may be labeled by replacement of a naturally occurring carbon, nitrogen, and/or hydrogen atoms by heavier “isotopes” of the same chemical elements. In particular embodiments, the amino acid is labeled with more than one different isotope (e.g., a combination of one or more of ¹³C heavy carbon, ²D heavy hydrogen (deuterium) and ¹⁵N heavy nitrogen). In one embodiment, the amino acid comprises serine and is labeled with 3 ¹³C heavy carbons, 3 ²D heavy hydrogens (deuteriums) and one ¹⁵N heavy nitrogen. In other embodiments, serine is derived from glycine, and glycine may be derived from threonine, such that glycine and/or threonine used in isotopically labeling yields labeled sphingolipids (though the yield may be less than when using serine).

In one embodiment, wherein the isotope-labeled amino acid comprises a non-essential amino acid, the method for the quantification of sphingolipids further comprises adding an amino acid synthesis inhibitor to the biological system. An “amino acid synthesis inhibitor,” as used herein, is a compound capable of preventing an organism's independent synthesis of an amino acid so that the isotope-labeled amino acid is the only amino acid available to the organism in the biological system. For example, because serine is a non-essential amino acid, a specific serine synthesis inhibitor can be added so that the only available serine is the isotopically labeled serine.

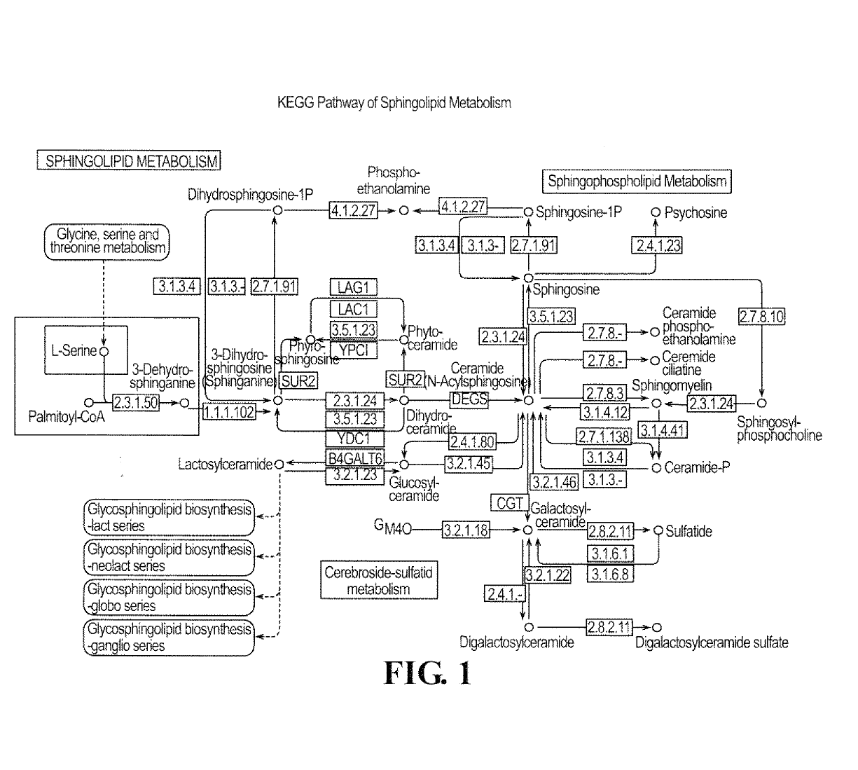

In one embodiment, the method for the quantification of sphingolipids comprises introducing the isotope-labeled amino acid into a biological system. As used herein, a “biological system” is a system comprising one or more sphingolipids. The biological system may be in vivo or in vitro. In one embodiment, the isotope-labeled amino acid comprises a serine that reacts in vitro with the compound palmitoyl Co-A as the starting point for the biosynthesis of sphingolipids, as illustrated in FIG. 1. Those skilled in the art will appreciate that any compound (i.e., a sphingolipid) that utilizes the isotope-labeled serine in its synthesis also will carry the isotope and therefore can be identified by its increase in mass and unique isotopic distribution.

In one embodiment, the method for the quantification of sphingolipids further comprising extracting the sphingolipids from the biological system. The extraction of the sphingolipids from the biological system may be conducted using any suitable method in the art. For example, in one embodiment the sphingolipids are extracted and separated from the biological system using liquid chromatography. In another embodiment, the sphingolipids are extracted from cells, tissue, or bodily fluids by solvent/solvent extraction and then separated by liquid chromatography prior to mass analysis. In some embodiments, the sphingolipids are extracted with supercritical fluid extraction (SFE). In other embodiments, the sphingolipids are extracted with thin layer chromatography (TLC), supercritical fluid chromatography (SFC), subcritical fluid chromatography, or enhanced fluid chromatography.

In one embodiment, the method for quantification of sphingolipids further comprises analysis of the quantities of sphingolipids after their extraction from the biological system. Any method known in the art for detection and measurement of the isotope-labeled sphingolipids may be used, non-limiting examples of which include mass spectrometry, Fourier transform ion cyclotron resonance mass spectrometry (FT-ICR MS), high resolution mass analysis (e.g., FT-ICR MS). In should be understood that any mass analysis technique having a resolution capable of baseline resolving the labeled and unlabeled glycosphingolipids may be used for quantification of the isotope-labeled sphingolipids.

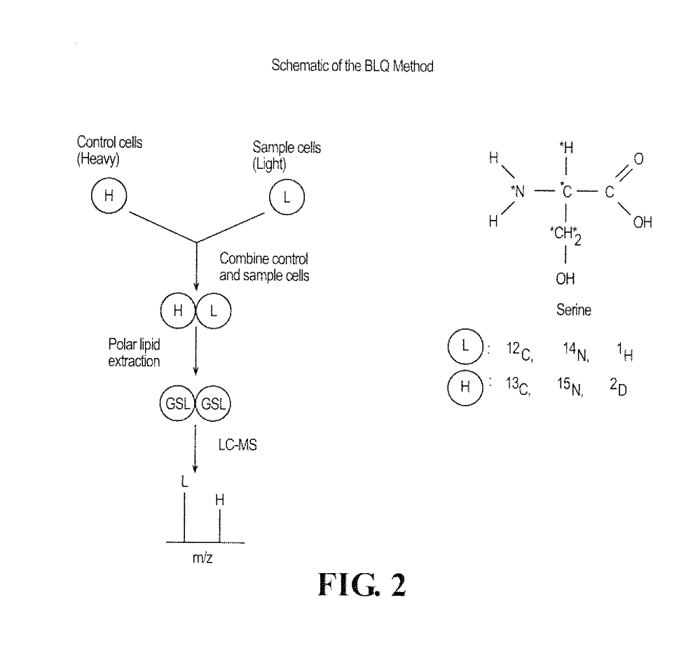

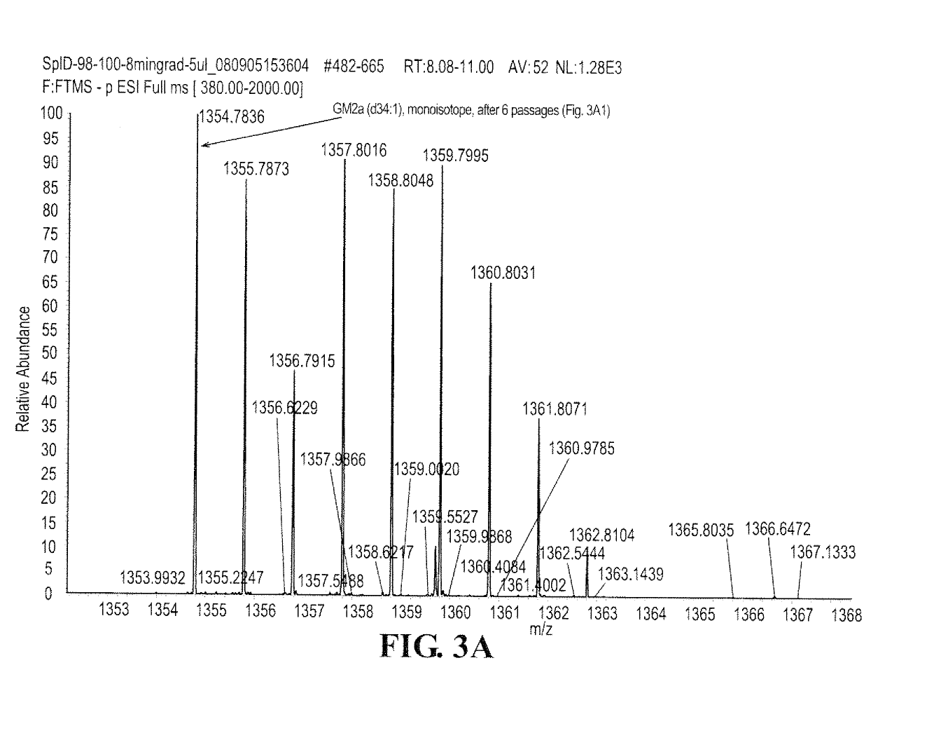

Not wishing to be bound by any theory, the sphingolipids that are extracted from the biological system will include both “light” and “heavy” sphingolipids based on type of isotope-labeled amino acid (“light” or “heavy”) that is introduced into the biological system. The quantities of sphingolipids in the extract can then be evaluated using the ratio of heavy sphingolipid ions to light sphingolipid ions detected using a mass spectrometer. For example, a serine loses one carbon (as CO₂) and one deuterium (in a hydrogen rearrangement) during the formation of the ceramide tail of the sphingolipid. The observed mass increase will be 5 atomic mass units (Daltons) for each labeled sphingolipid. Because the isotope-labeled amino acid and the naturally occurring amino-acid have essentially no chemical difference, the cells having labeled sphingolipids will behave substantially like the control cells grown in the presence of naturally-occurring serine. Thus, the sphingolipids can be identified as doublets separated in mass by 5 Daltons and the sphingolipids in the treated cells (“light”) can be accurately quantified relative to their respected control (“heavy”) (FIG. 2).

In one embodiment, the method comprises using high resolution Fourier transform ion cyclotron resonance mass spectrometry (FT-ICR MS) to detect the sphingolipids synthesized using isotope-labeled amino acids. FT-ICR MS provides both high-resolution and high mass accuracy and provides a means for determination of an ion's exact mass and isotopic distribution. Although FT-ICR MS has been used in various broad applications (e.g., to analyze chemical compounds, biological compounds, food, and petroleum), it is believed that the use of high-resolution mass spectrometry in the quantification of cellular sphingolipids by metabolic labeling provides particular advantages in evaluating the sphingolipid ion isotopic distributions.

The method provided herein provides significant advantages over prior art methods of qualitatively and quantitatively evaluating sphingolipids because the method is inherently quantitative. Both the labeled and unlabeled sphingolipid extracts from the biological system are handled together during all subsequent analysis. For example, the heavy sphingolipids are isotopes of the light sphingolipids and are chemically analogous. This chemical similarity provides the same extraction efficiency, same retention time (e.g., in liquid chromatography), same ionization efficiency (due to the same response to matrix effects), and same degree of scan-to-scan variation.

Not wishing to be bound by any theory, the metabolic labeling system provided herein also may be routinely applied in other areas of cell biology. For example, in one embodiment the metabolic labeling system can be adapted to provide a simple approach for the in vivo incorporation of stable isotopes into sphingolipids. In other embodiments, the labeling system can be used to follow isotope-labeled intermediates through specific biosynthetic pathways, thereby aiding in evaluation of the importance of specific pathways in the progression of disease states. In alternative embodiments, any biosynthetic pathway that utilizes a predetermined amino acid in its pathway can be traced with the isotopic labeling technique described. For example, serine feeds into several pathways, including the pathway for the formation of sphingolipids from serine and palmityl-CoA. Serine also feeds into a loop pathway that first forms hydroxypyruvate to glycerate to 3-phosphoglycerate to 3-phosphohydroxypryuvate to phosphoserine and back to serine. Intermediates in this pathway can feed into the glycolytic pathway and result in isotopically labeled lactate, which is the end product of glycolysis. In this way, it can be verified that the glycolytic pathway is being used for survival.

The present disclosure is further illustrated by the following examples, which are not to be construed in any way as imposing limitations upon the scope of the invention. On the contrary, it is to be clearly understood that resort may be had to various other embodiments, modifications, and equivalents thereof which, after reading the description therein, may suggest themselves to those skilled in the art without departing from the spirit of the present invention and/or the scope of the appended claims.

### EXAMPLES

### Example 1

L-serine was isotopically enriched in cell media to provide a heavy isotope-labeled serine with 3 ¹³C (97-99%), 3 ²D (97-99%) and 1 ¹⁵N (97-99%). Tissue culture cells were grown on normal L-serine and on isotopically enriched L-serine media. The two cultures were mixed and the lipids extracted and separated by liquid chromatography. The lipid extracts then were analyzed by mass spectrometry as illustrated schematically in FIG. 2. The sphingolipids appeared as a doublet separated in mass by the increase in mass of the isotopically labeled L-serine. The quantity of sphingolipids was then easily determined by comparison of the peak abundance of the non-labeled and labeled ions.

### Example 2

L-serine was isotopically enriched as described in Example 1. Tissue culture cells then were grown as described and sphingolipids were extracted and separated.

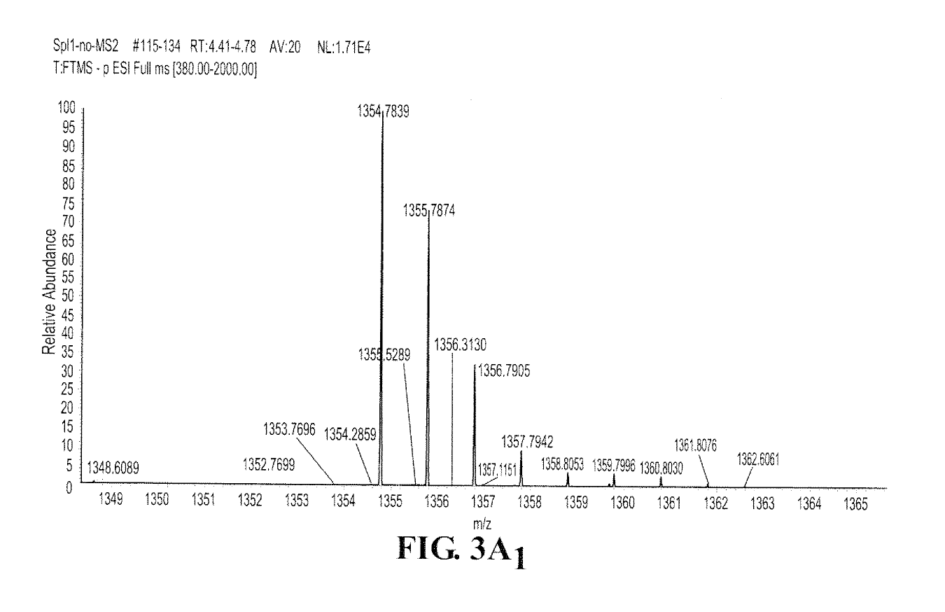

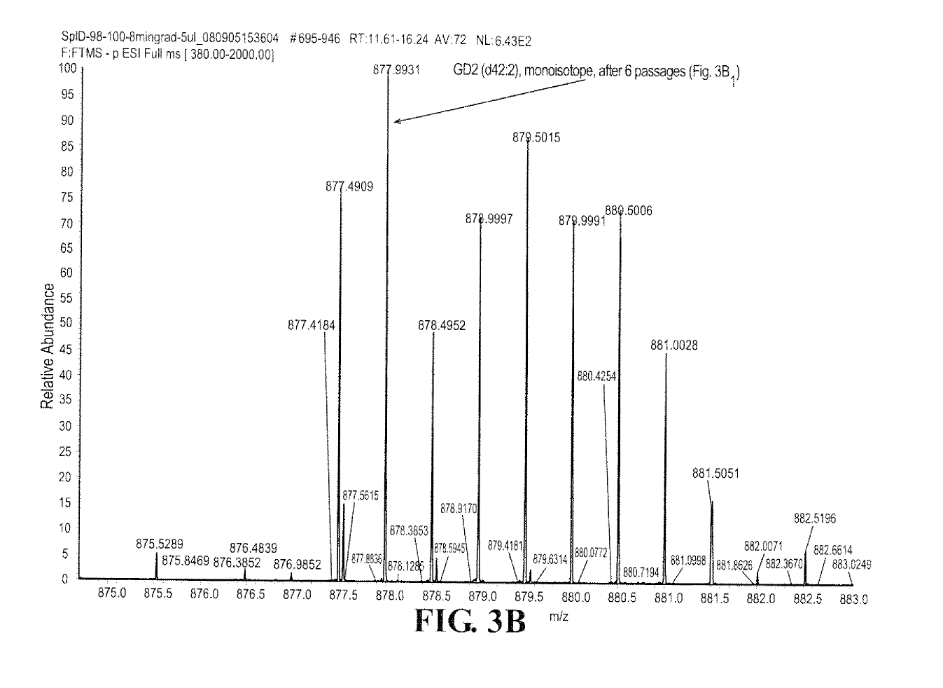

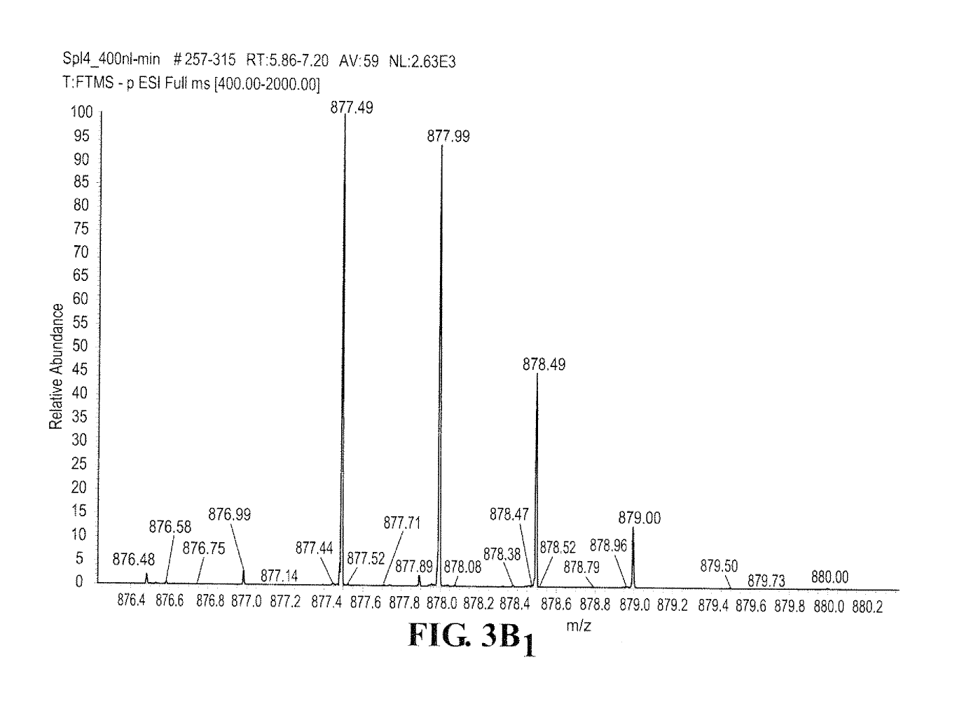

The mass spectra of the two polar lipids GM2α and GD2 are illustrated in FIGS. 3A and 3B, respectively. The inset spectra on the right depicts the isotopic distribution and mass of the polar lipid ions grown on normal isotope L-serine media. The mass spectra on the left depicts the isotopic distribution and mass of the polar lipid ions grown on isotopically enriched L-serine media. The polar lipid ions grown on the isotopically enriched L-serine showed an increase in mass and a unique isotopic distribution as compared to those grown on normal isotope L-serine media. These results demonstrate that the isotopically enriched L-serine was incorporated into the polar lipids and can be used for their quantification.

Publications cited herein and the materials for which they are cited are specifically incorporated herein by reference. Modifications and variations of the methods and devices described herein will be obvious to those skilled in the art from the foregoing detailed description. Such modifications and variations are intended to come within the scope of the appended claims.

We claim:

1. A method of quantifying endogenous sphingolipids in a biological system comprising:
preparing one or more isotope labeled amino acids;
introducing the isotope labeled amino acids into a biological system;
extracting and separating a sphingolipid-containing fraction from the biological system; and
quantifying the amount of endogenous sphingolipids in the biological system.

2. The method of claim 1, wherein the isotope-labeled amino acid comprises an isotope-labeled serine.

3. The method of claim 1, wherein the isotope-labeled amino acid comprises an isotope-labeled glycine.

4. The method of claim 1, wherein the one or more isotope-labeled amino acids comprise a heavy isotope-labeled amino acid and a light isotope-labeled amino acid.

5. The method of claim 1, wherein the amino acid comprises a non-essential amino acid the method further comprises adding an amino acid synthesis inhibitor into the biological system.

6. The method of claim 5, wherein the amino acid comprises a serine and the amino acid synthesis inhibitor comprises a serine inhibitor.

7. The method of claim 1, wherein the step of extracting and separating comprises liquid chromatography.

8. The method of claim 1, wherein the step of quantifying comprises mass spectrometry.

9. The method of claim 1, wherein the step of quantifying comprises Fourier transform ion cyclotron resonance mass spectrometry.

10. The method of claim 4, wherein the step of quantifying comprises comparing the ratio of a heavy sphingolipid-containing fraction and a light sphingolipid-containing fraction.

11. The method of claim 1, wherein the biological system is in vitro.

12. The method of claim 1, wherein the biological system is in vivo.

13. A kit for the quantification of cellular sphingolipids by metabolic labeling comprising one or more isotope-labeled amino acids.

14. The kit of claim 13, wherein the isotope-labeled amino acid comprises an isotope-labeled serine.

15. The kit of claim 13, wherein the isotope-labeled amino acid comprises an isotope-labeled glycine.

16. The kit of claim 13, wherein the one or more isotope-labeled amino acids comprise a heavy isotope-labeled amino acid and a light isotope-labeled amino acid.

17. The kit of claim 13, wherein the isotope-labeled amino acid comprises a non-essential amino acid and the system further comprises an amino acid synthesis inhibitor.

18. The kit of claim 17, wherein the amino acid comprises a serine and the amino acid synthesis inhibitor comprises a serine inhibitor.
